(19) United States
(12) Patent Application Publication
(10) Pub. No.: US 2010/0015174 A1
(43) Pub. Date: Jan. 21, 2010

(54) METHOD TO CONTROL DENGUE VIRUSES IN HUMANS BY PICOLINIC ACID AND DERIVATIVES THEREOF

(76) Inventors: Jose Alberto Fernandez-Pol, Chesterfield, MO (US); Sebastian Fernandez-Pol, Chesterfield, MO (US)

Correspondence Address: CHARLES C. MCCLOSKEY, 763 S. NEW BALLAS ROAD STE. 170, ST. LOUIS, MO, 63141, US

(21) Appl. No.: 12/175,277

(22) Filed: Jul. 17, 2008

(51) Int. Cl. A61K 31/4402; A61K 36/06; A61P 31/12

(52) U.S. Cl. 424/195.16; 514/354

(57) ABSTRACT

A method treats and then prevents a virus for afflicting an animal or a human as a metalloprotein mediates the virus. The method administers systemically a therapeutic pharmacological agent of picolinic acid either singly or with interferons, chemokines or cytokines to fight dengue fever virus. The picolinic acid inactivates the metalloprotein that allows replication of the virus. The picolinic acid has the structure of:

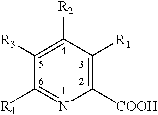

where R1, R2, R3, and R4 are mutually exclusive. The viral proteins disintegrate by macrophage proteolytic enzymes stimulated by the picolinic acid.

### BACKGROUND OF THE INVENTION

The method to control dengue viruses in humans by picolinic acid and derivatives thereof relates to pharmaceuticals and their usage, and more specifically to a pharmaceutical that raises and maintains tryptophan levels to summon macrophages.

The virus etiology of dengue fever was discovered during 1944 when Dr, Albert Sabin isolated the first dengue viruses from soldiers in India, New Guinea and Hawaii. The data show that the dengue virus, not chikungunya, was responsible for the majority of epidemics in the past 40 years, generally transmitted through the Aedes aegypti mosquito. A dramatic increase in urbanization following World War II created ideal conditions for increased transmission of urban mosquito-born diseases. These changes, associated with an increased movement of people within and between countries via air travel resulted in the emergence of dengue hemorrhagic fever (DHF) and dengue shock syndrome (DSS). Since 1993, dengue fever has become the most important arbovirus disease of humans because of over 2 billion people at risk in a belt of countries around the tropics that widens continuously as temperatures warm away from the equator. Dengue viruses have a worldwide distribution in the tropics and the viruses are endemic in most urban centers of the tropics with transmission occurring year round. See Kyle, J. and Harris, E., Global Spread and Persistence of Dengue, Annu Rev Microbiol, April 2008.

### The Dengue Virus

### Taxonomy and Classification

Dengue viruses belong to the family Flaviviridae, genus Flavivirus. There are four serotypes: DEN-1, DEN-2, DEN-3 and DEN-4 that belong to a larger heterogeneous group of viruses called arboviruses. This ecological classification implies that transmission between vertebrate hosts including human depends upon hematophagous arthropod vectors.

### Structure of Dengue Viruses

Dengue viruses consist of a single-stranded RNA genome surrounded by an icosahedral nucleocapside, covered by a lipid envelope derived from the host cell membrane from which the virus buds. The mature virion contains three structural proteins: the nucleocapside (core protein), a membrane associated protein and the envelop protein. Over 60 antigenically related flaviviruses, include the prototype, yellow fever, St, Louis encephalitis and others.

### Host Range and Virus Propagation

There are three natural hosts for dengue viruses: Aedes mosquitoes, humans and lower primates. Viremia in humans may last 2 to 12 days. The titers ranging from undetectable to 10⁸ Infectious doses (MID₅₀/MI).

Dengue viruses are known to cause clinical illness and disease only in humans. Mammalian cell lines commonly used to study Dengue include Vero (monkey kidney), BHK-21 (baby hamster kidney) and FRhL (fetal rhesus lung).

### Genetics

Oligonucleotide fingerprinting, restriction enzyme, primer extension sequencing and nucleotide sequence comparison using polymerase change reaction have all been used to study genetic variation among dengue viruses. In general, viruses in the same geographic region during the same time period show genetic homogeneity. The rate of mutation change is very low, generally about 1.4% annually.

### Evolution

Biologically, dengue viruses have highly adapted to their mosquito hosts for transovarian transmission. With the clearing of forests and jungles, and the development of human settlements, Dengue viruses move out of the jungle and into the rural and city environments, where they still are and are transmitted to human by mosquitoes such as Aedes Albopictus.

### Serologic Relationships and Variability.

Dengue viruses share common morphology, genomic structure and antigenic determinants with over 60 other flaviviruses. Hemagglutination inhibition (HI), complement fixation (CF) and plaque reduction neutralization (PRNT) tests. Because all flaviviruses share common antigenic determinants, identification of individual family members using these test is difficult. Dengue virus complex are most accurately and easily identify with an indirect immunofluorescent antibody assay using sera-type specific monoclonal antibodies which react to epitopes on the structure of the protein.

Both antigenic and biologic variation among dengue viruses have been documented. DEN-3 viruses (Caribbean, southern coast of the US and South Pacific islands in the 1960's) were found to be antigenically distinct from the prototype and the Asian strains in baby mice and mosquitoes. DEN-4 virus, introduced in the Caribbean in 1981, was distinct from DN-4 viruses from Asia.

When dengue was reintroduced in Pacific Islands in 1971 after 25 year absence, epidemics occurred in numerous islands. Marked variation in disease severity, viremia levels, and epidemic duration were found. Some of the epidemics were explosive and others were mild but some caused hemorrhagic disease and high levels of morbidity. The diversity of the spectrum of sign and symptoms was clearly attributed at urban changes and not to strain variation.

### Epidemiology

Dengue viruses exist in nature in three basic maintenance cycles. The primitive forest cycle involves canopy-dwelling mosquitoes and lower primates. A rural cycle involves mosquitoes and humans. The urban cycle which is the most important epidemiologically and in public health impact because of death and morbidity, involves highly domesticated Aedes Aegypti mosquito and humans. The viruses inhabit essentially all large urban centers of the tropics with epidemics occurring at periodic intervals due to the changes in temperature, rain, humidity and pollution from global warming and the pollution produced by human industries and services.

A combination of the following factors has made most if not all tropical cities highly permissive for dengue transmission by Aedes Aegypti:

1) increased urbanization in the tropics, changing lifestyles (vacationers) and lack of effective mosquito control; comparison of epidemics in urban regions of the Republic of Brazil and climate conditions appears to make no difference with similar conditions in the Southern U.S., suggesting that it is not a question of if, but when, a Dengue epidemic will hit the Southern U.S. or even northern states;

2) because of air travel, in the past 20 years, the movement of the Dengue virus has dramatically increased within and between regions, resulting in increased epidemic activity and hyperendemicity, and the spread and increased incidence of severe and fatal forms of the disease, dengue hemorrhagic fever and dengue shock syndrome, hereinafter DHF/DSS.

While considered only a disease of Southeast Asia, DHF/DSS has spread in epidemic form to west Asia, all of the Pacific islands, and the Americas in numerous urban regions. In the Americas, the incidence of dengue expands in less than 10 years.

The most pathogenic and scientific support upholds the hypothesis that the increased movement of the virus and the new human hosts of different genetic configurations has resulted in the development of an ominous set of dengue viruses. These viruses have increased virulence and multiple hyperendemicity (denoting circulation of multiple virus serotypes in the urban community) which increase the chances of new dengue viruses in the community. This combination may result in multiple dengue diseases in one host, even a person.

### Transmission and Tissue Tropism

Dengue viruses are only transmitted by the bite of an Infected mosquito that prefers to feed on humans than in animals. Interestingly, the mosquito has a nearly undetectable bite and is very restless. The slightest movement makes the mosquito interrupt the feeding and fly away.

In addition to transmitting the virus to humans or lower primates, the female mosquito may also transmit the virus through the eggs to her offspring. This mechanism cannot be underestimated for the indefinite and permanent natural maintenance cycles of dengue viruses in rural, forest and urban areas.

### Definitions

Within this specification, the term “response modifier” is intended to encompass all of the intended functions of the invention and method including antiviral, anti-infective, anti-inflammatory, anti-cancer, vaccine, and like medicinal effects.

The term “anti-Infective” is intended to include antibacterial, anti-fungal, anti-parasitic functions, and actions against any other Infective agent or organisms including viruses not encompassed by the term “antiviral”.

The term “anti-Inflammatory” is intended to include an Inflammatory response modifier, including production of stress proteins (e.g., heat shock proteins), white blood cell infiltration, intravenous and intra-arterial thrombotic Inflammatory reactions, symptoms of dengue hemorrhagic fever (DHF), increases in vascular permeability accompanying dengue shock syndrome (DSS), and like reactions within people.

### Human Phagocytic Monocytes and the Dengue Virus

Human phagocytic monocytes and their derivatives, the macrophages, as in FIG. 1a, are the primary sites of dengue virus replication, following injection into a human host from a vector, and highly valuable targets for the control of dengue. The virus has been isolated from many other tissues including the liver, lungs, kidneys, lymph nodes, stomach and intestine, as in FIG. 1b but it is unclear if the virus replicates in these parenchymal tissues. The virus replicates in the phagocytes of these tissues, but these are completely different host cells in relationship to parenchymal cells that perform the specific functions of these organs but do not protect the tissue. The tissue function protection against dengue falls under the monocytes and macrophages plus an array of cytokines. Interestingly, the dengue virus can replicate in vascular cells and perhaps in the bone marrow cells, or the precursors of monocytes, as in FIG. 2

### Pathogenicity

The main pathogenicity of dengue virus is capillary leak syndrome which if not corrected quickly, may lead to hypovolemic shock and death. The cause of this pathogenetic mechanism is related to an immune enhancement phenomenon where the infecting virus complexes with non-neutralizing dengue antibody. This complex enhances the Infection of mononuclear tissue phagocytes. As a result of the non-neutralizing dengue antibodies, the monocytic phagocytes switch to a new mode of activation: production of vasoactive mediators (cytokines and other peptides) that increase vascular permeability. Loss of plasma from the vascular compartment leads a progressive, mild, transient, severe, or prolonged leak of plasma that later leads to severe shock and death.

Patients Infected with dengue viruses may experience severe and uncontrolled bleeding, usually in the upper gastrointestinal (GI) tract. This syndrome involves disseminated intravascular coagulation and thrombocytopenia. Based on recent studies of intravascular coagulation, perhaps nicotinic acid, picolinic acid, or both together could be highly effective in controlling this syndrome, see Fernandez-Pol, experimentation with thrombosis and pyridine carboxylates, unpublished. Picolinic acid has the follow representation:

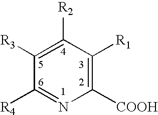

### Clinical Features of Infection

Dengue Infection causes a spectrum of illnesses in a human ranging from clinically in-apparent to severe and hemorrhagic disease. Encephalophathy appears secondary to general deterioration of patient conditions. Rush, joint pains, nausea and vomiting and lymphadenopathy are common and indicate the disease as highly debilitating. Thrombocytopenia and hemoconcentration constantly afflict patients. Dengue virus also causes a variety of neurologic disorders, including headache, dizziness, hysteria and depression.

### Pathology and Histopathology

This section is accompanied by various figures and appended references to provide detailed information on the molecular biology developed in the last few years to expand the specific aims concerning treatment and prevention of Dengue.

The pathology of dengue virus Infection has a poor understanding because systematic post-mortem studies have not occurred on patients representing all types of clinical expression and the wide spectrum of the disease.

The major pathophysiologic abnormalities in classical DHF/DSS are an increase in vascular permeability (produced by cytokinins and other peptides), damaging endothelial cells and leakage of plasma. Because of this noxious attack of the virus, patients usually have serious effusions in pleural, abdominal cavities, joints and, in extreme cases, the leakage of blood across the blood/brain barrier. In such a symptom, blood vessels also become leak prone and produce fatal encephalophathy. It is not believed that the virus crosses the blood/brain barrier.

Destructive Inflammatory vascular lesions have not been found with any regularity. However, some swelling and occasional necrosis and apoptosis have been observed in endothelial cells as well as pericytes, and some perivascular edema.

Studies on patients with a fatal outcome has demonstrated focal necrosis of the hepatic cells. From the point of view of viral invasion, the liver also shows Councilman bodies and hyaline necrosis of Kupffer cells (which are macrophages derived from blood monocytes). Changes in the kidneys in phagocytic histiocyte also suggest an immune-complex type of glomerulonephritis. The production of bone marrow elements of all lineages also dwindles but which improve when the patient becomes afebrile.

Biopsy studies of skin rash have demonstrated perivascular edema with infiltration of lymphocytes and monocytes which eventually are transformed in Langherans cells (monocytes of blood origin), as in FIG. 3.

### Immune Response

Persons Infected with dengue viruses produce immunoglobulin M (IgM) and IgG antibodies, detected in approximately 5 to 7 days after onset of the disease (primary Infection). In contrast to IgM antibodies that disappear in about 90 days after initiation of disease, IgG antibodies persist for at least 50 years or for the life of the patient.

### Prevention and Control of Dengue

The options available for prevention and control of Dengue and DEN/DHF are limited, no vaccines exists at present. Genetically engineered vaccines now in a experimental phase offer hope, but it will take at least 10 years to develop an effective vaccine.

Unfortunately the ability to control Aedes Aegypti is limited at present time. All of the control methods using, such as ULV, applications of insecticides to kill mosquitoes in the field and urban areas have failed. The ULV method has no effect on transmission of dengue viruses. The only method of controlling the mosquitoes reduces the source of larval habitats of the mosquito. Larval habitats generally occur in domestic environments where most urban transmission occurs.

### Future Perspectives

Continued urbanization of the tropics, global warming, increased air travel and the difficulty in controlling mosquitoes have been the dramatic factors responsible for the incidence, increased virulence and geographic expansion of DHF/DSS. This rapid trend will continue with dengue in general and DHF/DSS becoming a major cause of increase expenditures in massive hospitalizations and death among children in South America, North America and Africa, unless public health efforts and in particular scientific advances undergo rapid implementation. The present invention accomplishes the tasks of reversing the dengue disease and its various incapacitating and deadly effects.

Since Pasteur, vaccines have played a major role in controlling and eradicating viral and bacterial diseases. Ultimately, development of an economical tetravalent vaccine holds the apparently greater promise for prevention and control.

### SUMMARY OF THE INVENTION

The present invention provides a method where a systemic preparation of picolinic acid, its derivatives or analogs, containing approximately 1% to 100% active ingredient, may be administered orally, intravenously, intramuscularly or by any acceptable route for the treatment of viral systemic infections such as Dengue fever and other arboviruses. In one embodiment, picolinic acid prepared in 00 gelatin capsules at 500 mg per capsule controls certain lymphomas, and viral diseases such as Herpes simplex labialis, herpes zoster and papilloma viruses in humans. Within the teachings of the invention, the safe and effective daily systemic dose may range from approximately 250 mg to approximately 6 grams a day, and the most preferred dose being within approximately 500 mg to approximately 2000 mg per day. The invention also includes picolinic acid being provided in combination with interferons, nicotinamide, nicotinic acid, and other biological response modifiers.

### BREIF DESCRIPTION OF THE DRAWINGS

In referring to the drawings,

FIG. 1a illustrates a macrophage;

FIG. 1b illustrates sources in the body of various macrophages;

FIG. 2 illustrates the development of a macrophage; and,

FIG. 3 shows one macrophage, a langerhans cell, within epidermal cells.

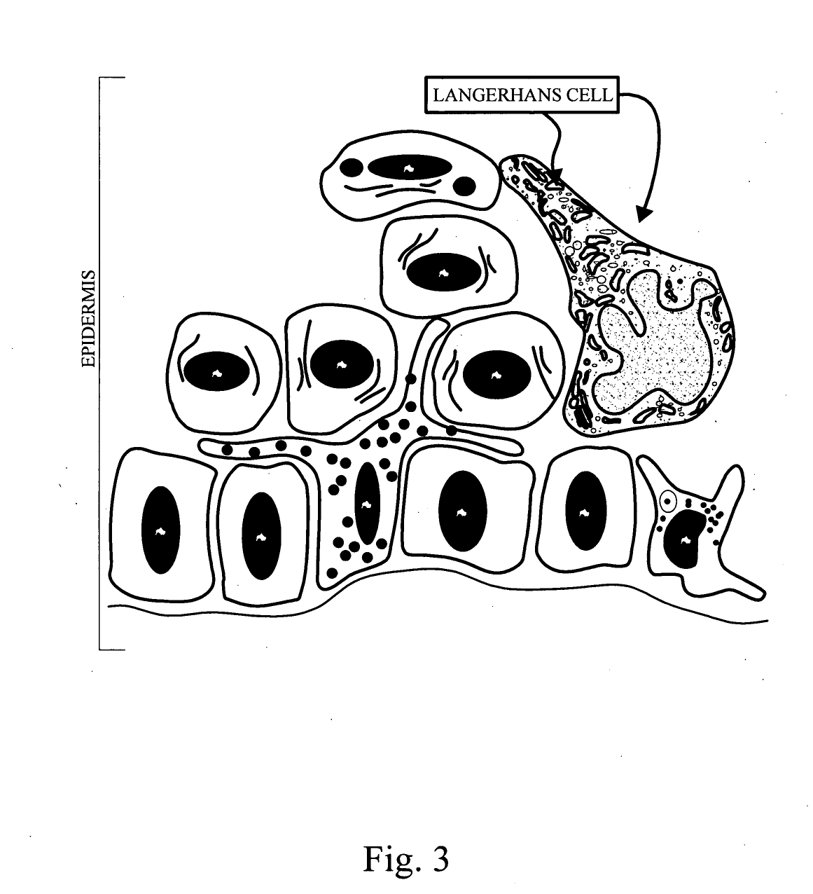

### BREIF DESCRIPTION OF THE TABLES

In the remainder of the specification, tables are provided showing laboratory conditions and results from various experiments described in the examples that follow:

Table 1 interferon properties;

Table 2 picolinic acid effect on retroviruses;

Table 3 sindbis replicon inhibition at various concentrations;

Table 4 lightcycler operating conditions;

Table 5 picolinic acid effect on hepatits B;

Table 6 fusaric acid effect on hepatitis B;

Table 7 chart of Tables 5, 6;

Table 8 fusaric acid effect on VZV;

Table 9 chart of Table 8;

Table 10 picolinic acid and fusaric acid effects upon BVDV;

Table 12 picolinic acid molecule and its analogs along with substitution data; and,

Table 13 nicotinic acid, nicotinimide, and NAD and NADP molecules.

### DETAILED DESCRIPTION OF THE INVENTION

Advances in molecular biology now attack the viruses directly in their transcriptional machinery and activate the macrophages widely distributed throughout tissues by pirydine carboxylates to destroy numerous existing viruses and to change the configuration of the viral nucleoproteins and other proteins which effectively become antigens for auto vaccination. Inclusive of the four types of Dengue viruses and numerous other tested viruses, the present invention offers a “quantum jump” for prevention and control of viral diseases with non-toxic antiviral agents that also are capable of conferring immunity against critical proteins against replication of Dengue viruses and numerous other RNA or DNA animal viruses.

The immune system employs a variety of strategies to combat virus Infection. These can be divided into innate or “non-specific” defenses, which include interferons, natural killer cells (NK) cells, blood monocytes and macrophages [derived from blood monocytes] and adaptive or specific immunity that involves T cell lymphocytes and antibodies.

Immunological defenses, “non-specific” or “specific,” concentrate at strategic areas in the body where entry of a virus occurs following millions of years of evolution. For example, IgA at all mucosal surfaces of the body functions by blocking virus adsorption and penetration. Macrophages are located through the body in various tissues and interferons are produced by most in response to viral Infection. Once the primary line of defenses is breached, viruses and viral antigens (many processed by macrophages to generate antibodies) become directed into the lymphoid system where they encounter T and B lymphocytes. From such interactions, antiviral T cells are generated that target and rapidly destroy Infected cells, and antiviral antibodies are produced that protect the host from viremia. In the following section, the protection by the immune system will be contrasted with the evasion strategies employed by the viruses, particularly the Dengue viruses.

### Innate Immune Defenses

Because the first line of defense mobilizes instantaneously, in a matter of minutes or hours, the innate defenses may determine the balance between the host resistant responses to the virus or wide-speed systemic Infection. Restricting the early infection is the task of interferons alpha and beta, natural killer cells, and macrophages. The Inflammatory response, that follows this initial encounter with viruses, signals other immune cells to indicate the location of the virus to the specific immune system, which is rapidly activated, and becomes effective after a few days to a week. At the end of this period, T cells will converge on the site of Infection attracted by cytokines and the antibodies are active in limiting the virus spread.

### Antiviral Properties of Interferons

Interferons, the first line of defense against viruses, have two families (IFN):

type I, which includes IFN-alpha (leukocyte produce) encoded by about 20 genes located on chromosome 9, IFN-beta (fibroblast-type) encoded by a single gene on chromosome 9, IFN-omega and IFN-tau (trophoblastic); and,

type II, which includes IFN-gamma (immune), encoded by a single human gene on human chromosome 12.

IFN-gamma can exert direct antiviral activity, a pivotal role in the activation of the immune cell system: activation of macrophages and the induction of other anti-viral cytokines and defense molecules. It is also associated with the terminal differentiation of B and T lymphocytes. IFN-gamma is strongly stimulated by alpha-picolinic acid and derivatives thereof (Picolinic acids produced by macrophages) to produce more IFN-gamma.

### NK Cells in Antiviral Immunity

NK cells are large granular lymphocytes distinct from T and B lymphocytes and can be activated directly by interleukin 2 (IL-2). Picolinic acid and derivatives thereof also strongly stimulate NK cells which become active and destroy viruses, cancer cells and also virally-transformed cells. When activated, NK cells produce numerous cytokines involved in antiviral immunity and tumor destruction, including IFN-gamma, Tumor Necrosis Factor (TNF), Transforming growth factor-beta (TGF-beta-1), which inhibits and destroys tumors, and numerous other cytokines including GM-CSF (Granulocyte-macrophage colony stimulating factor). A subtype of NK cells stimulated by picolinic acids, denoted NK1.1, CD8-positive cells, have been shown to have important regulatory functions in the immunological response.

Functionally, NK cells are characterized by their ability to kill tumor cell lines in vitro and in mice. Varesio et al. has further shown that the killing ability and production of cytokines can be greatly increased by picolinic acid. NK cells can also recognize IgG antibody-bound to cell surfaces of virus-infected cells via the CD16 receptor and kill target cells. This important process is known as antibody-dependent cell cytotoxicity: a highly efficient mechanisms for activating NK cells that rapidly destroy virus-infected cells. Cell killing is mediated by a performing-dependent system. NK cells are rapidly mobilized following viral Infection, stimulated by picolinic acid and derivatives thereof. They are detected at the site of Infection within 2 days of virus entry. Viruses with pronounced susceptibility to NK cells in vivo include Herpes viruses of all types. Herpes viruses have been extensively studied for more than 20 years with greater than 98% effective in aborting the disease and destroying the viruses in humans, animals and tissue culture.

In humans, NK cells play an important role in immunity to herpes virus Infection, including cytomegalovirus, and varicella-zoster. When patients having outbreaks of these viruses undergo treatment with preparations of picolinic acid and derivatives thereof, both the Herpes labialis and the Herpes-Zoster viruses are destroyed in less than 2 hours and the pain disappears within less than three hours, if the patients are treated early, during the onset of the disease.

The Key Role of Macrophages in Immunity to Viruses and Picolinic Acid

Macrophages are strategically placed through the body as first line of defense against Infectious agents. The inventors have developed experimental antiviral pharmaceutical agents, based on picolinic acids and derivatives thereof, that greatly and directly increase the potent antiviral activity of macrophages by two main mechanisms: 1) activating macrophages and 2) destroying the zinc finger proteins and other metalloproteins of the viruses. Fernandez-Pol, 1995. These two mechanisms render the virus unable to replicate. Then the viruses disintegrate by proteolysis enzymes and depending upon the degree of damage of the virus to the macrophage, this cell either recovers or enters into apoptosis. This process eliminates both the virus Infected macrophage and the intracellular viruses thus effectively stopping the viral Infection. The picolinic acids are also very potent antiviral agents in the extracellular spaces by attacking intra-viral zinc finger proteins such as the nucleoprotein Np-7 of the AIDS virus and disintegrating the virus.

Macrophage antiviral activity includes intracellular and extracellular defense mechanisms that can be pharmacologically enhanced greatly by agents such as picolinic acids and derivatives thereof. Macrophages are the major producers of IFN-alpha found in the blood stream following viral Infection. Other antiviral molecules produced by macrophages include:
1. Tumor Necrosis Factor-alpha, which functions by inducing gene coding for 2′5′-A synthetase and Mx, and lysing virus-infected cells, by apoptosis;
2. The enzyme Arginase, which is induced following IFN-gamma activation of macrophages and effectively aborts HSV Infection;
3. The enzyme nitric acid synthase, which is induced following IFN-gamma activation of macrophages and leads to generation of nitric oxide which inhibits the replication of vaccinia virus and HSV;
4. The cessation of the production of picolinic acid by viruses in macrophages, effectively neutralizes the capacity of macrophages to destroy viruses and activate the immunological defense systems. The inhibition of picolinic acid production in macrophages is most likely due to the inhibition of the substrate (beta-muconic acid) of picolinic acid, and/or picolinic acid carboxylase, which leads to the formation of picolinic acid resulting in the destruction of zinc finger proteins of the invading viruses, and activates the antiviral activities of macrophages to produces IFN-gamma and other antiviral cytokines.

Tryptophan metabolism inhibition by viral Infection is another viral strategy to shut off antiviral picolinic acid production. The pathogenic virus has taken control of viral replication inside the cell by disabling the powerful macrophage defense systems. However, combining IFN-gamma and picolinic acid yields a potent inhibitor of replication of the J2 recombinant retrovirus that has v-raf and v-myc oncogenes present in macrophages. Blasi, et al. 1988. While picolinic acid operates in synergism with IFN-gamma, thus activating macrophages to express tumoricidal actions. Varesio, et al. 1990. And further, interleukin-4 also inhibits the costimulation of interleukin-2 and the picolinic acid and IFN-gamma activated macrophages. Cox et al. 1991. As a catabolite of tryptophan, picolinic acid also inhibits the in vitro and in vivo growth of cell lines and transformed those line to have heightened sensitivity to picolinic acid and thus interference with transition metal ion uptake. Fernandez-Pol, 1977. Picolinic acid also improves hormonal stimulation and the AMP response to PGE1, or isoproterenol, by several fold. Picolinic acid further increases the GTP dependent activities of the adenylate cyclase system. Johnson and Fernandez-Pol 1977.
5. For many viruses, the hostile intracellular environment of a phagolysosome (producing superoxide radicals and other deleterious chemical radicals), leads to a loss of Infectivity and viral destruction. These processes are of two types: 1) oxygen-dependent and 2) oxygen independent mechanisms that contribute to the inactivation of numerous viruses which shows that viruses can be targeted for intracellular destruction by phagocytosis of Infected cells.
6. Picolinic acid and Fenton Reactions. The Applicants have demonstrated in vitro and in vivo that chelate picolinic acid-Fe²⁺ can produce Fenton reactions which produce superoxide ions and other oxygen free radicals that are deleterious for the virus and the Infected cells. Thus, this compound can destroy virally Infected cells without any significant effect in normal cells, which do not allow the penetration of the picolinic acid-Fe²⁺.
7. When activated by Picolinic acid, macrophages can also kill virus Infected-cells by direct contact. Despite the hostile nature of macrophages, some viruses, such as Dengue virus, may exploit these cells for growth and survival. These phenomena occur because of the capacity of certain viruses to shut off critical defense mechanism of macrophages, such as picolinic acids. Thus, the pharmacological use of picolinic acid and derivatives thereof as anti-Dengue virus pharmacological agents to replenish endogenous picolinic acid shut off by the virus appears effective and has already defeated other viral diseases, such as Herpes, Herpes-Zoster varicella and others, in tissue culture. Picolinic derivatives include radicals about the pyridine ring from the butyl group, carboxyl group, ethyl group, hydrogen, isobutyl group, isopentyl group, isopropyl group, methyl group, neopentyl group, pentyl group, propyl group, secondary butyl group, and the tertiary butyl group.

### The Role of Cytokines in Viral Infection

Cytokines induced during viral Infections have numerous functions such as modulation of the immune response. This section summarizes, in Table 1, the human interferons, their genes, proteins produced, their producer cells and their effects which could act in concert with picolinic acid.

TABLE 1

| Name | Genes | Proteins | Producer Cells | Effect |
|---|---|---|---|---|
| IFN-α | >20 | Non-glycosylated | Lymphoid cells | Antiviral state |
| | No introns | 166aa | Macrophages | ISG induction |
| | Chromosome 9 | Monomer | | MHC 1 induction |
| IFN-β | 1 | N-glycosylated | Fibroblasts | Antiviral state |
| | No introns | 166aa | Epithelial cells | ISG induction |
| | Chromosome 9 | Dimer | Macrophages | MHC 1 induction |
| IFN-ω | 5 | N-glycosylated | Leucocytes | Presumably similar to |
| | No introns | 172aa | Trophoblasts | IFN-α |
| | Chromosome 9 | | | |
| IFN-γ | 1 | N-glycosylated | T cells | 2′,5′-OAS induction |
| | 3 introns | 146aa | Macrophages | IL-1 enhancement |
| | Chromosome 12 | Tetramer | NK cells | MHC I induction |
| | | | | MHC II induction |

| | |
|---|---|
| IFN | interferon |
| ISG | interferon stimulated gene |
| MHC | major histocompatibility complex |
| 2′,5′-OAS | 2′,5′-oligoadenylate synthetase |

### Picolinic Acid, a Catabolite of L-Tryptophan, Activates Macrophage Effector Functions

The effector functions of a macrophage lead to tumoricidal, microbicidal and antivirial activities of mononuclear phagocytes. The tryptophan catabolite picolinic acid (PA) has the ability to modulate the expression of chemokines in macrophages. PA is a potent activator of the inflammatory chemokines macrophage inflammatory protein (MIP)-1-alpha and MIP-1-beta Mrna expression macrophages through a novo protein synthesis-dependent process (Bosco et al., J. Immunology, 2000, 164: 3283-3291). Iron chelation may be involved in MIP induction by PA because iron sulfate inhibited the process and the iron chelating desferroxmine, induced MIP expression. Thus, Bosco et al. discovered the existence of a new pathway leading to inflammation initiated tryptophan metabolism that communicates with the immune system by the production of PA. The increase of intracellular PA concentration in macrophages then leads to secretion of chemokines by macrophages. These findings establish the importance of PA as an activator of macrophage pro-inflammatory functions, providing evidence that PA can be biologically active without one or more co-stimulatory agents. (Bosco et al, J. Immunology, 2000, 164: 3283-3291).

The attraction of inflammatory cell populations to sites of injury or Infection is controlled by the local secretion of chemotactic signals, often released by macrophages. The chemokine super-family of small chemotactic proteins contains numerous members which have been characterized chemically and biologically. Chemokines are produced by both immune and non-immune cells in response to inflammatory stimuli or tissue damage produce by microbes such as numerous positive strand viruses, including Dengue, hepatitis C, Sinbis and others. The chemokines are involved in a large array of immuno-regulatory, Inflammatory and anti-viral activities, including leukocyte migration and activation.

The primary synthetic source for chemokines is activated mononuclear phagocytes, which are critical mediators of cellular immunity against Infections and tumors. The mononuclear phagocytes release the effector molecules, or recruit T lymphocytes and NK cells, to target tissues though the release of chemokines.

### Amino Acid Catabolites Signal for Mononuclear Phagocyte Functioning

For instance, the metabolism of two essential amino acids, L-typtophan (L-TRP) and L-arginine, have been implicated in antimicrobial activity, T cell tolerance, and in biological effects of IFN-gamma. The inducible enzyme controlling L-TRP catabolic pathway [indoleamine 2,3-dioxygenase (IDO)] is present in an active form in inflammatory tissues. L-TRP catabolism, initiated by IDO, leads to the production of biologically active molecules. Among the most important antiviral agent, PA is the end-product of L-TRP degradation. PA is endowed with important immuno-modulatory properties including activation of mononuclear phagocyte effector functions.

PA is a potent co-stimulus for the induction of macrophage mediated cytotoxicity. PA also contributes to the microbicidal and antiviral activity of macrophages in vitro and in vivo.

PA, in combination with IFN-gamma, inhibits retrovirus expression in macrophages both in vitro and in vivo, and activates the transcriptional activity of the inducible NO synthase gene, leading to production of NO, a potent effector molecule identify in the expression of macrophage microbicidal activities.

### Function of Macrophages as Antiviral Agents

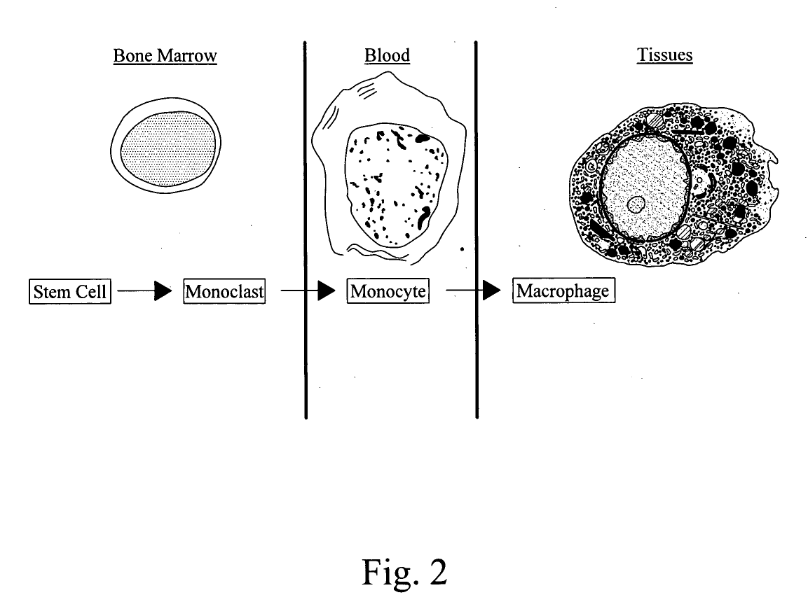

Macrophages are cells derived from monocytes produced in the bone marrow, as in FIG. 2. Monocyte-derived macrophages (MDM) migrate and reside in numerous tissues. The developmental differentiation of macrophages from progenitor cells in the bone marrow to tissue of residence is controlled by numerous hormones denoted cytokines (Hashimoto et al, 1999). Gene expression during this complex process has involved about 35,000 different transcripts expressed in human blood monocytes and macrophages after induction by the cytokines GM-CSF or M-CSF.

Blood monocytes migrate to various tissue targets in response to the release of various chemotactic factors due to Infection or cell damage. In such tissues, a complex differentiation of the monocytes takes place which results in a macrophage population denoted “resident macrophages.” These macrophages can be characterized in subsets, and in IFN-gamma-stimulated macrophages by using monoclonal antibodies, (Leenen et al; 1994).

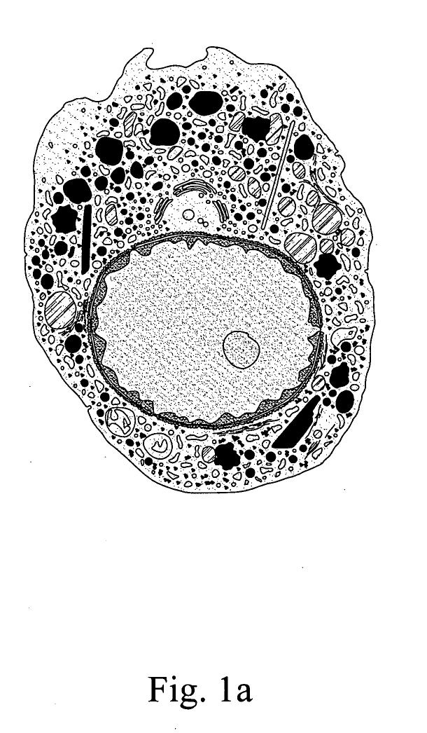

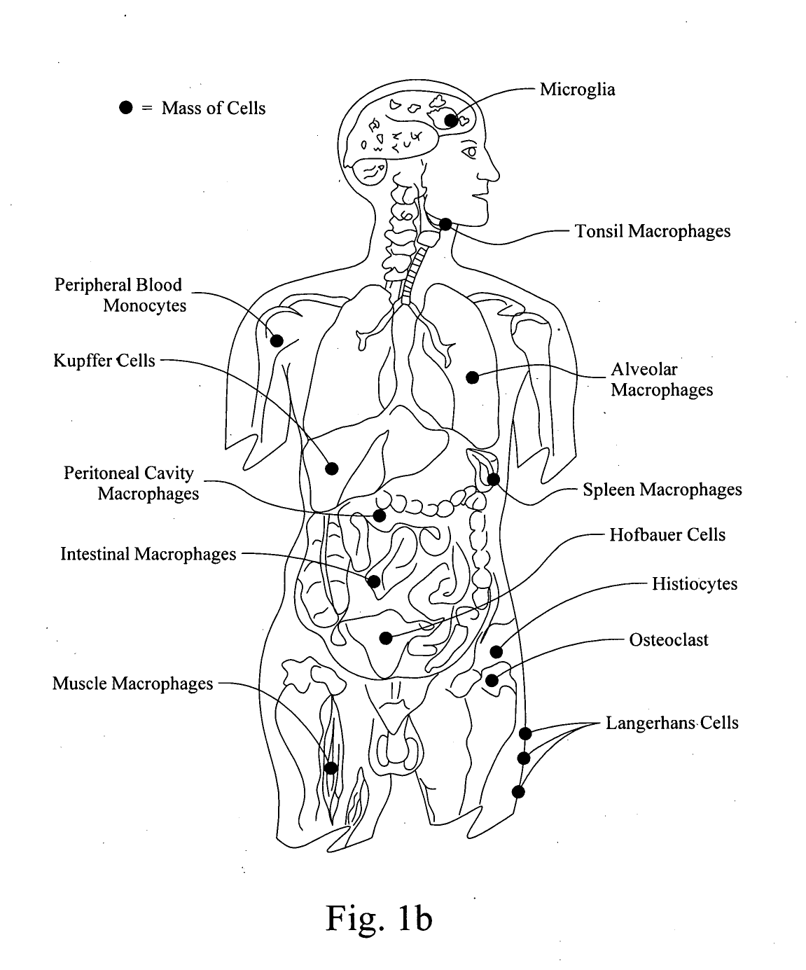

Resident macrophages (also denoted Normal Macrophages as in FIG. 1a) are found in numerous tissues, as in FIG. 1b, such as liver, lung, lymph nodes, spleen, bone marrow, synovial fluids, skin, brain, lipophages (which are lipid-laden macrophages, Siderophages (which accumulate insoluble precipitates of Fe³⁺, including connective tissue contains resident macrophages and are denoted histiocytes. Other resident macrophages are identified in FIG. 1b.

### Langherans Cells: Targets for Dengue Viruses

One important type of resident skin macrophage is known as Langherans cells. Langherans cells are the preferred target for mosquitoes carrying all four types of Dengue virus. In these cells, the production of intracellular virus after Infection paralleled that for secreted Dengue virus (Pryor et al; Am J Trop Med Hyg, 65, 2001, 427-343). The local populations of macrophages are replenished by proliferation of resident progenitor macrophages and the continuous influx of monocytes from the blood while the cell damage or Infection lasts.

Inflammatory macrophages are derived from monocytes, have analogous properties and are present in various exudates such as pericarditis, peritonitis, synovial fluids among others.

Activated macrophages, including M1 macrophages, are resident macrophages that have been activated in response to LPS, cytokines or IFN-gamma. These cells participate in immune reactions to antiviral agents or other microbes. The gene expression patterns of activated macrophages confers them the capacity to secrete mediators of immune reactions.

Thus, the different types of macrophages above and the M1 macrophages are a heterogeneous population of cells differing in 1) origins and differentiation stage, and 2) response to viral Infection and to micro-environmental influences by chemokines and cytokines. The versatility of these cells displays a spectrum of responses to microbial invaders in a myriad of damaging circumstances. Activated resident macrophages have proliferative capacity, show potent phagocytic activity, and respond to various biological and chemical stimuli, but in the absence of noxious forces the M1 macrophages remain relatively quiescent with respect to immunological and secretor functions utilized to maintain tissue integrity.

Macrophages are critical in all phases of the immune response, and since the populations of macrophages are functionally distinct, they provide for an impressive, powerful and flexible response. They play a key role in host defenses against intracellular parasitic micro-organisms such as bacteria and viruses and also in the control of tumor cell growth. Macrophages can kill extracellular targets by the use of antibody-dependent cellular cytotoxicity

Macrophages activated by pathogenic prokaryotic or eukaryotic cells are a critical link in the following defense processes: 1) presentation of antigens to T-cells and, 2) production of chemokines and cytokines. These macrophage properties interact with other cells in synergism and the cells become bioactive.

Activated macrophages secrete a large array of cytokines, growth factors, and other small molecule chemicals which participate in autocrine and paracrine activities that affect many other cell types. Because activated macrophages are also a primary cell target in vivo for Dengue Virus (DV) Infection, replication, and propagation, these macrophage responses and functions must have an antiviral role to control the DV invasion.

### Picolinic Acid, Macrophage Activation and Inhibition of Pathogenic Virus Expression

PA, in combination with Interferon-gamma (IFN-gamma), inhibits retrovirus expression in macrophages, both in vivo and in vitro and triggers the transcriptional activation of inducible isoform of the nitric oxide (NO) synthase gene, stimulating production of NO as in Table 2, a major effector molecule implicated in the expression of macrophage tumoricidal and microbicidal activities (Varesio et al., 2000).

TABLE 2
Picolinic Acid

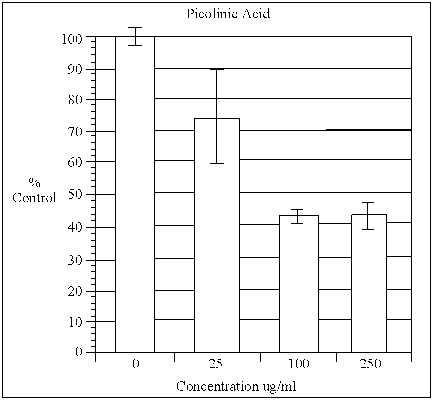

Varesio et al. also investigated the GG2EE transformed by the recombinant J2 retrovirus carrying the v-raf and v-myc oncogenes to study the effects of biological response modifiers (BRM), picolinic acid (PA) and/or IFN-gamma, Varesio et al. et al (J. Immunology, 141, 2153-2157, 1988). J2 retrovirus expression was not affected by picolinic acid or IFN-gamma alone but it was dramatically decreased by the picolinic acid plus IFN-gamma. These results indicate that transformed GG2EE macrophages can be inhibited by the combination of PA plus IFN-gamma. In addition, the combination resulted in a potent inhibition of J2 retrovirus mRNA expression in the cells, inhibition of cell growth and activated the tumoricidal capacity of macrophages (Varesio et al., 1991).

Picolinic acid is a potent co-stimulator for the induction of tumoricidal activity and the production of L-arginine-dependent reactive nitrogen intermediates (RNI) in macrophages. The results indicate that IFN-gamma and picolinic acid can regulate nitric-oxide synthase, or transcription and mRNA expression and nitric oxide transcription. (Mellilo et al, J. Biol. Chem., 269, 8128-8133, 1994). Further results demonstrate that picolinic acid is a potent biologic macrophage secondary signal, in the activation of IFN-gamma-prime macrophages and indicate the existence of an autocrine effect mediated by picolinic acid. (Varesio et al., 1990).

Picolinic acid, a naturally occurring product of tryptophan catabolism is a biological response modifier, endowed with a variety of effects on transition metal ion traffic, cell cycle, bacterial, viral and fungal growth and host immune responses mediated by macrophages. Picolinic acid is an immune-response modifier inducing a variety of chemokines and cytokines, including interferons, tumor necrosis factor, and interleukins. In animal model systems and in vitro, picolinic acid has demonstrated antiviral activity, for example in Herpes viruses, and immune modulatory activities. In addition, inactive macrophages respond to picolinic acid with macrophage activation, a mechanism of elimination of viruses. (Fernandez-Pol, 2001).

It is conceivable that in addition to the effects of picolinic acid described above, picolinic acid and structurally related derivatives inactivate zinc finger proteins (ZFP). Picolinic acid renders the viral ZFPs inactive by ejecting Zn²⁺ from the zinc finger domain. Picolinic acid inhibits viral replication in transformed cells and normal cells treated with Picolinic acid enter into a temporarily dormant state with no toxicity observed. The action of Picolinic acid on ZFPs matches the results showing activation of macrophages by Picolinic acid. (Fernandez-Pol, 2001).

### Arboviruses: Togaviridae and Flaviviridae

Studies on the antiviral activity of picolinic acid and derivatives thereof on virulence of Arboviruses face many problems because of the availability of human monocyte-derived macrophages Infected with dengue in which the virus can replicate and the lack of a suitable animal model for dengue Infection. The evaluation of antiviral efficacies, mode of action, toxicity and toxicity profiles of picolinic acid using cultured cells—those at sites of virus replication in humans Infected with Togaviridae or Flaviviridae—may provide important models of the antiviral effects of picolinic acid on dengue viruses.

Some of the viruses, described in this section, were used to evaluate the broad spectrum of antiviral efficacies of picolinic acid and fusaric acid against human cytomegalovirus (CMV), human varicella-zoster virus (VZV), human hepatitis B virus (HBV), and bovine viral diarrhea virus (BVDV). Before presenting the examples a few basic concepts on Togaviridae and Flaviviridae will be described to facilitate the interpretation of the antiviral effects of picolinic acid and derivatives thereof.

The group of arboviruses is a heterogeneous group of viruses in seven taxonomic families, including Togaviridae and Flaviviridae. Arboviruses have a global distribution but are mostly found in tropical regions. Because important limiting factors are temperature, rainfall patterns, and urbanization, they are rapidly emerging diseases.

Togaviridae is a family of enveloped, positive (+) stranded RNA viruses containing many agents responsible for important diseases in animals and humans. Togaviridae was initially established with two genera, Alphavirus and Flavivirus (Wildy, 1971). The family Flaviviridae contains three genera: Flavivirus, Pestivirus and hepatitis C viruses. The virion structure contains a nucleocapside protein complexed with single-stranded positive (+) sense RNA. Target cells are usually of the mononuclear phagocyte lineage.

Treatment is supportive and restricted to non specific measures, including maintenance of fluids and electrolyte balances and replacement of significant amounts of blood lost through hemorrhage.

In the case of Dengue, no lasting cross-protective immunity exists, the immunity lasts at the most for 3 months (Sabin, 1952). No good animal model for Dengue hemorrhagic fever (DHF) exists at present. Currently, prevention depends on mosquito control without vaccines.

Despite the evidence demonstrating the importance of PA for the activation of macrophages alone or in combination with cytokines and the capacity of PA to destroy viral zinc finger proteins-essential for viral replication and mutation of the zinc finger domain, no information exists on PA and derivatives thereof on Dengue viruses, or other Togaviridae and Flaviviridae (Fernandez-Pol, 2001).

### EXAMPLE 1

### Evaluation of Antiviral Activities of Picolinic Acid and Fusaric Acid Against Human Hepatitis C Virus

The sequence of the HCV genome shares several features with other positive (+) strand RNA viruses. HCV is most like members of the Flaviviridae in that the genomes of Flaviviridae, dengue fever virus, and yellow fever virus, have similar organization and have common features with HCV. They have a similar genome size to HCV (yellow fever virus: 10862 bases (Ruce et al., 1985) and compared with 9379 of G+HCV (Coho et al, 1991)). Certain features of HCV are shared with Pestiviruses. Structurally, HCV is more like the pestiviruses than the flaviviruses. The difference is related to extensive glycosylated proteins in the virus envelope, in contrast to flaviviruses envelope glycoproteins that contains a few sites for N-linked glycosylation. The genome of HCV is single-stranded RNA of positive (protein coding) polarity. The genomic RNA of positive-strand RNA viruses is Infectious.

Many aspects of the pathology of HCV Infection result from the host immune response. HCV is capable of cytophathic replication in cell types outside the liver, including monocytes and macrophages. Replication can also occur in certain lymphoid cells.

Using a highly strand specific Polymerase chain reaction (PCR) method, initiation of transcription is understood for some positive-strand RNA viruses. Picornaviridae, togaviruses and flaviviruses contain a single strand of positive-strand RNA that has mRNA activity. Detection of HCV genome is indispensable for the accurate diagnosis, the complete characterization of HCV Infection in a patient and for evaluation of antiviral drugs. The PCR test is the method of choice for these studies.

Here we determine the effects of Picolinic acid and Fusaric Acid on HCV replicon cell lines. The HCV replicons (qRTCPCR/iCycler) were contained in the Ava.5 cell line tested. Normalization by OD and serial dilutions were done in the untreated cell line. The experimental design was as follows: cells were grown in 12 well cell culture plates; the compounds interaction was done for 24 hours; at extraction, cell confluence was 75%; and RNA was extracted using Qiagen Rneasy extraction kit.

Table 3 shows the HCV Replicon Data for both picolinic acid and fusaric acid at various concentrations. The results show that picolinic acid at 250 ug/mL shows approximately 60% inhibition of HCV replicon. By OD, no overt cytotoxicity was observed. At a concentration of 100 ug/mL, fusaric acid show about 60% inhibition of HCV replicon and by OD, no overt cytotoxicity was observed.

### EXAMPLE 2

### Antiviral Effects of Picolinic Acid and Fusaric Acid on Sindbis Virus Replicons

Sindbis virus has been designated as the prototype Alphavirus and belongs to the Togaviridae family. Sindbis is a positive-single stranded DNA virus related to HCV.

Here we determine the effects of Picolinic acid and Fusaric Acid on Sindbis replicon in Huh7B/Sindbis cells. The Sindbis replicons were studied by qRTCPCR/iCycler. Normalization by OD and serial dilutions were done in the untreated cell line. The experimental design was as follows: cells were grown in 12 well cell culture plates; the compounds interaction was done for 24 hours; at extraction, cell confluence was 75%; and RNA was extracted using Qiagen Rneasy extraction kit.

Table 3 shows the Sinbis Replicon data for both picolinic acid and fusaric acid at various concentrations. The results show greater than 90% inhibition of Sindbis replicon at all concentrations of picolinic acid. By OD, no overt cytotoxicity was observed. Fusaric acid show >85% inhibition of Sindbis replicon at all concentrations tested. No overt cytotoxicity was observed by OD.

TABLE 3
Sindbis Replicon

| | No Drug | IFN100 | IFN10 | STDS | PA 400 | PA 300 | PA 200 | PA 100 | FA 100 | FA 50 | FA 25 | FA 5 |
|---|---|---|---|---|---|---|---|---|---|---|---|---|
| Sindbis | 20.5 | 23.5 | 23.1 | 20.4 | 24.0 | 24.3 | 24.1 | 24.1 | 22.2 | 22.8 | 23.0 | 22.7 |
| | 21.0 | 24.0 | 22.7 | 22.1 | 23.5 | 23.7 | 22.9 | 23.7 | 23.8 | 23.4 | 22.7 | 23.3 |
| | 20.3 | 23.5 | 22.6 | 23.4 | 23.2 | 21.6 | 24.2 | 23.5 | 24.0 | 22.7 | 22.4 | 23.1 |
| | 20.1 | 23.5 | 22.9 | 25.8 | 24.3 | 23.7 | 24.1 | 23.6 | 23.9 | 24.2 | 23.5 | 21.7 |
| AVE | 20.5 | 23.6 | 22.8 | | 23.7 | 23.3 | 23.8 | 23.7 | 23.5 | 23.3 | 22.9 | 22.7 |
| SD | 0.4 | 0.2 | 0.2 | | 0.5 | 1.2 | 0.6 | 0.3 | 0.8 | 0.7 | 0.5 | 0.7 |
| % CV | 1.7 | 1.0 | 0.9 | | 2.1 | 5.1 | 2.6 | 1.1 | 3.6 | 2.9 | 2.1 | 3.1 |
| % CTRL | 100.0 | 7.0 | 12.1 | | 6.7 | 11.3 | 6.5 | 6.5 | 8.8 | 9.3 | 11.6 | 14.0 |
| % CV | 14.0 | 1.1 | 1.5 | | 2.2 | 11.3 | 2.8 | 1.2 | 6.3 | 3.7 | 3.2 | 6.6 |

### EXAMPLE 3

### Evaluation of Antiviral Efficacies of Picolinic Acid and Fusaric Acid Against Human Hepatitis B Virus (HBV)

The endogenous template of HBV virus is a circular partial double stranded DNA. It was found that the DNA strand of negative polarity was transcribed inside the core particle from an encapsidated RNA-template, indicating a similarity to retroviruses (Summers and Mason 1982). Although the genome organization, biology and replication of hepadnaviruses (HBV) and reverse transcription define HBV as virus order “retroviral,” Hepadnavirire are quite different from those of the Retroviridae, the common strategy has also been denoted Para retroviruses. Because HBV DNA in infected hepatocytes is not integrated, it remains as an episomal mini-chromosome (Newbold et al. 1995). The HBV polymerase is, in contrast to retroviral polymerase, devoid of an integrated domain. Integration of the HVB DNA into the host genome is not part of the hepadnaviral life cycle, in contrast to the retroviruses.

Here we evaluate the antiviral efficacy of picolinic acid and fusaric acid against human hepatitis B virus (HBV). HepG2.2.15 cells, a cell line that was stably transfected with HBV genome was used to study the antiviral effects of PA and FA against HBV. HepG2.2.15 cells were cultured under standard conditions. HepG2.2.15 cells were seeded in a 96-well plate at 2×10⁴ cells/well and grown for 24 hours. The medium was replaced with 100 uL of medium containing PA at increasing concentrations (0, 10, 100, 250, and 500 ug/mL). The concentrations of FU used were 0, 1, 10, 30, 60, and 100 ug/mL. The cells were incubated at 37° C. for 9 days. The control medium and the medium containing the antivirals were changed every 24 hours. At day 9, the culture medium containing the released HBV virions was collected, the cells washed with PBS, and lysed with lysis buffer. All samples were stored at −70° C. until analyzed.

### DNA Extraction

HBV DNA from drug-treated HepG2.2.2.15 cell culture medium and cell lysates was extracted using QIAamp DNA mini kit, according to manufacturer's specifications.

Detection of HBV DNA by Real-Time PCR (Lightcycler assay)

PCR reaction was performed in 20 uL, comprising the following compounds: dH₂O; 25 mM MgCl₂; 10 uM sense primer; 10 uM antisense primer; LightCycler Probe 1 (fluorescent-labeled, 5 uM); LightCycler Probe 2 (LC Red 640-labeled, 10 uM); LC FastStart DNA Master Hyb Probes; and Uracil DNA glycosylase (1 uL).

PCR mix was aliquoted and 5 uL DNA template was then added to the PCR mix. A range of HBV plasmid DNA (1×10⁸, 1×10⁷, 1×10⁶, [ . . . ], 1×10², 25 copies) were used as standards for quantitative analysis. The cycling conditions are shown in Table 4.

TABLE 4
The cycling conditions of LightCycler assay

| | | | | | Acquisition |
|---|---|---|---|---|---|
| Program | # cycles | Target Temp | Incubation Time | Temp Transition | Mode |
| Denaturation | 1 | 95° C. | 450 sec | 20° C./sec | None |
| PCR | 45 | 95° C. | 5 sec | 20° C./sec | None |
| | | 60° C. | 15 sec | 20° C./sec | None |
| | | 72° C. | 10 sec | 20° C./sec | None |
| Melt | | 98° C. | 0 sec | 20° C./sec | None |
| | | 40° C. | 15 sec | 20° C./sec | None |
| | | 85° C. | 0 sec | 0.2° C./sec | Continuous |
| Cool | 1 | 40° C. | 30 sec | 20° C./sec | None |

### RESULTS OF EXAMPLE 3

HepG2.2.2.15 cell line was created by transfection of full-length HBV genome into HepG2 cells, and continuously secreted HBV virions into the medium. This cell line is the prototype cell line used in studying the efficacy of antiviral compounds against HBV Infection.

To study the antiviral activities of PA and FA against HBV, HepG2.2.15 cells were incubated with increasing concentrations of test compounds for 9 days with medium change daily. At day 9, the medium containing secreted HBV virions was collected and used for extraction of extracellular virus DNA. The cells were lysed by 0.5% SDS and used for extraction of intracellular virus DNA. DNA was extracted using Qiagen column and then quantitatively detected by real-time PCR assay. The results showed that PA at concentrations of 250 ug/mL inhibited released HBV DNA by 72.3% and intracellular HBV DNA by 79.4% with overall EC₅₀ of approximately 139 ug/mL, see Table 5. FA at concentration of 60 ug/mL inhibited the released of HBV DNA by 76.5% and intracellular HBV DNA by 28% with overall EC₅₀ greater than 60 ug/mL, see Table 6. Then Table 7 shows the antiviral effects of PA (A) and FA (B) on HBV after 9-days of treatment on the cell line HepG2.2.15 cells.

TABLE 5
Effect of PA on HBV after 9-day treatment of HepG2.2.15 cells

| | | Concentrations (μg/ml) | | | |
|---|---|---|---|---|---|
| | | 0 | 10 | 100 | 250 |
| Released | Copies × 10⁵ | 12.8 | 8.556 | 3.822 | 3.542 |
| HBV DNA | % of control | 100.0 | 66.8 | 29.9 | 27.7 |
| Intracellular | Copies × 10⁵ | 74.15 | 97.89 | 42.91 | 15.29 |
| HBV DNA | % of control | 100.0 | 132.0 | 57.9 | 20.6 |

Note:
500 ug/ml was slightly toxic to HepG2.2.15 cells.

TABLE 6
Effect of FA on HBV after 9-day treatment of HepG2.2.15 cells

| | | Concentrations (μg/ml) | | | | |
|---|---|---|---|---|---|---|
| | | 0 | 1 | 10 | 30 | 60 |
| Released | Copies × | 15.69 | 6.035 | 7.184 | 6.747 | 3.681 |
| virus DNA | 10⁵ | | | | | |
| | % of | 100.0 | 38.5 | 45.8 | 43.0 | 23.5 |
| | control | | | | | |
| Intracellular | Copies × | 1 | 1.287 | 1.146 | 1.163 | 0.72 |
| virus DNA | 10⁷ | | | | | |
| | % of | 100.0 | 128.7 | 114.6 | 116.3 | 72.0 |
| | control | | | | | |

Note:
100 ug/ml was toxic to HepG2.2.15 cells.

TABLE 7

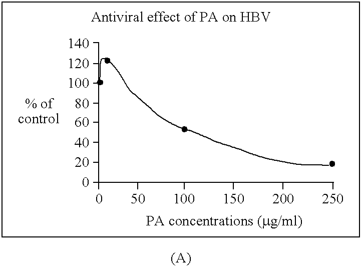

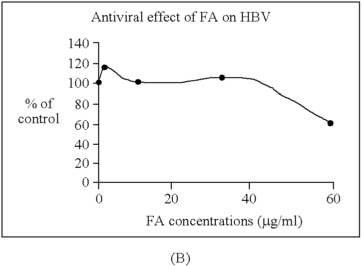

### EXAMPLE 4

### Evaluation of Antiviral Activities of Picolinic Acid and Fusaric Acid Against Varicella-Zoster Virus (VZV)

VZV Ellen cell line, from ATCC, and was used as the reference strain in the susceptibility tests. Human fibroblast cell line HEL-299 (ATCC) was used in the virus yield reduction assay.

### Virus Yield Reduction Assay

HEL-299 cells (90% confluent) in a 24-well plate were washed and subsequently infected with VZV Ellen strain at a moi of 0.5 pfu/cell for 2 h at 37° C. Following removal of viral inoculums, the Infected cells were washed and covered with culture medium final concentrations of PA (final concentrations were: 0, 1, 10, 100, 250, and 500 ug/mL) or FA (final concentrations: 0, 0.1, 1, 10, and 60 ug/mL) for 3 days, see Tables 8, 9. At the end of the drug treatment, the culture medium was collected for extraction of released virus DNA. The Infected cells were washed with PBS and lyzed with 400 uL of SDS lysis buffer (0.5% SDS, 10 mM Tris.HCl pH 8.0, 1 mM EDTA). The cells were used for extraction of intracellular VZV virus DNA.

TABLE 8
Antiviral effect of FA on VZV determined
by quantitation of DNA copy numbers using real-time
PCR (% of control)

| | FA (μg/ml) | | | | |
|---|---|---|---|---|---|
| | 0 | 1 | 10 | 30 | 60 |
| Total VZV DNA | 969115 | 753340 | 685520 | 103405 | 66230 |
| copies (mean) | | | | | |
| % of control | 100 | 77.7 | 70.7 | 10.7 | 6.8 |

EC₅₀ = 14 μg/ml
EC₉₀ = 41 μg/ml

TABLE 9

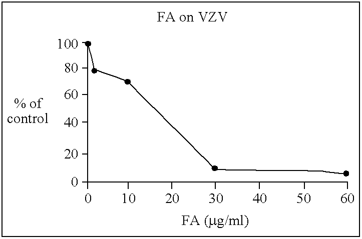

### VZV DNA Extraction

For extraction of the released virus DNA, 200 uL of drug-treated culture medium was digested with 500 ug/mL of proteinase K at 37° C. overnight in a buffer containing 0.01 M Tris.HCl (pH 7.8), 0.001 M EDTA and 0.5% SDS. The VZV DNA was purified by extractions with phenol/chloroform and chloroform. The VZV DNA was precipitated by addition of 1:10 vol of 3 M sodium acetate (pH 5.2) and 2.5 volumes of absolute ethanol. Following washing with 70% ethanol, the DNA was air-dried and dissolved in 20 uL sterile water.

For purification of the intracellular VZV DNA, the cell lysates were directly digested with 500 ug/mL of proteinase K at 37° C. overnight. The DNA was then extracted by phenol/chloroform and precipitated with ethanol as described above. After washing with 75% ethanol, the DNA was dissolved in 20 uL of sterile water.

### Detection of VZV DNA by Real-Time PCR (Lightcycler Assay)

PCR reaction was performed in 20 uL, comprising the following compounds: dH₂O; 25 mM MgCl₂; 10 uM sense primer; 10 uM antisense primer; LightCycler Probe 1 (fluorescent-labeled, 5 uM); LightCycler Probe 2 (LC Red 640-labeled, 10 uM); LC FastStart DNA Master Hyb Probes; and Uracil DNA glycosylase (1 uL).

PCR mix was aliquoted and 5 uL DNA template was then added to the PCR mix. A VZV DNA standard was serially diluted (1×10⁷, 1×10⁶, 1×10⁵, [ . . . ], 1×10², 10 copies) and were used as standards for quantitative analysis. The cycling conditions are shown in Table 9.

The data was generated from triplicate experiments. The effect of a test compound at various concentrations is expressed as % of control (the mean DNA copies in drug-treated wells/the mean DNA copies in control wells without test compound). The 50% effective concentrations (EC₅₀: effective concentration giving 50% reduction of DNA copies) were calculated with a computer program.

### RESULTS OF EXAMPLE 4

The primers and probes for the Lightcycler assay were previously published (Espy et al., Diagnosis of varicella-zoster virus Infection in the clinical laboratory by LightCycler PCR. J. Clin Microbiol 38, 3187-3189, 2000). Previous experiments show no significant toxicity to HEL cells when PA was used at concentration of 500 ug/mL and for FA at concentration of 60 ug/mL. The culture medium was collected for the detection of released virion DNA and the cells were lysed for the detection of intracellular virus DNA. The antiviral efficacies were determined by quantitative detection of total VZV DNA copy numbers using Lightcycler assay. The results showed that PA was active against VZV replication with EC₅₀ and EC₉₀ approximately 89 ug/mL and 505 ug/mL, respectively. Anti-VZV activity was also seen for FA with EC₅₀ approximately 14 ug/mL and EC₉₀ approximately 41 ug/mL, see infra Tables 8, 9.

### EXAMPLE 5

### Evaluation of Antiviral Efficacies of Picolinic Acid and Fusaric Acid Against Bovine Viral Diarrhea Virus (BVDV)

BVDV Trangie strain, a well characterized non-cytopathic Australian isolate, was used in this study. The virus was cultured in MDBK cells (ATCC), and titred by immunoperoxidase staining.

### Drug Treatment

80% confluent MDBK cells were infected with BVDV at a moi of 0.01 at 37° C. for 1 h. After viral inoculums was removed, Infected cells were washed with PBS and then incubated with culture medium containing PA at concentrations of 0, 10, 100, 250, 500, and 1000 ug/mL or FA at concentrations of 0, 1, 10, 30, 60, and 100 ug/mL at 37° C. for 3 days. The culture medium was then collected and stored at −70° C. The cells were harvested by one frozen/thawed cycle and centrifugation to pellet debris. The supernatant was collected and stored at −70° C.

### Virus Titration by Immunoperoxidase Staining

PA of FA-treated cultures were 10-fold serially diluted, 50 uL of each dilution was added to 80% confluent MDBK cells in a 96-well plate, and incubated at 37° C. for 1 h. The innoculum was removed by washing with PBS once. The Infected cells were covered with 200 uL of 0.5% methylcellulose in culture medium and incubated at 37° C. for 2 days. After washing with PBS, the cells were fixed with 200 uL of 5% formalin in PBS for 10 min at room temperature. Subsequently, they were washed with 0.05% Tween −20/dH₂O three times. Then, the cells were incubated with 50 uL mixture of three monoclonal antibodies against BVDV NS3 for 80 min at 37° C. in a humidified box. The cells were then washed as above and incubated with 50 uL of rabbit anti-mouse IgG Peroxidase-conjugate (DAKO) diluted 1:200 in 1% gelatin/PBS for 90 min at 37° C. in a humidified box. After final washes as described above, 200 uL of 3-amino-9-ethyl-carbazole was added to each well and incubated at room temperature for 30 min until the cells were stained.

### RESULTS OF EXAMPLE 5

The studies of antiviral agents against HCV have been hampered by their inability to propagate the virus efficiently in cell culture. Comparative studies indicate that HCV is closely related to Flaviviruses (i.e. dengue virus and more closely to pestiviruses (i.e. bovine viral diarrhea virus (BVDV). In fact, pestiviruses have been used as a model and surrogate for HCV. Information obtain by the BVDV virus is also useful to understand flavivirus replication such as dengue viruses.

The BVDV closely resembles HCV persistent Infection. After Infection with BVDV cultured MBDK were treated with different concentrations of PA or FA for 3 days. The antiviral effect was determined by virus yield reduction assays (i.e. staining using monoclonal antibodies against NS3, or titration of intracellular and extracellular virus by immunoperoxidase staining.

The results, see Tables 10, 11, showed that after treatment of Infected cells for 3 days PA at a concentration of 250 ug/mL reduced BVDV virus yield by 76% (EC₅₀=60 ug/mL. This indicates that both PA and FA are active against BVDV.

TABLE 10

| | Concentrations of PA (μg/ml) | | | | |
|---|---|---|---|---|---|
| | 0 | 10 | 100 | 250 | 500 |
| BVDV virus yield × 10⁵ (pfu/ml) | 4.1 | 3.2 | 2.5 | 1.0 | 0.35 |
| % of control | 100.0 | 78.0 | 61.0 | 24.4 | 8.5 |
| | Concentrations of FA (μg/ml) | | | | |
| | 0 | 1 | 10 | 30 | 60 |
| BVDV virus yield × 10⁵ (pfu/ml) | 3.0 | 2.4 | 1.8 | 2.5 | 1.5 |
| % of control | 100.0 | 80.0 | 60.0 | 83.3 | 50.0 |

TABLE 11

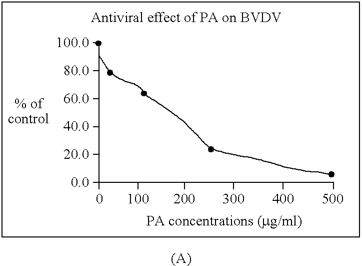

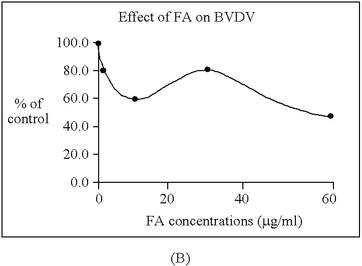

### EXAMPLE 6

### Evaluation of Antiviral Efficacies of Picolinic Acid and Fusaric Acid Against Cytomegalovirus (CMV)

CMV AD 169 strain (ATCC) was used in the experiments. A CMV susceptible cell line, human fibroblast cells HEL-299 (ATCC), was used in the plaque reduction assays.

### Drug Treatment

90% confluent HEL-299 cells were washed and then Infected with CMV AD169 at 0.5 moi for 2 h at 37° C. Following removal of viral inoculums, the Infected cells were washed and exposed to different concentrations of PA (final concentrations: 0, 10, 100, 250, 500, and 1000 ug/mL) or FA (final concentrations: 0, 1, 10, 30, 60, and 100 ug/mL for 3 days. The culture medium was collected for titration of released virions and the cells were harvested for titration of intracellular virions.

### Virus Titration

90% confluent HEL-299 cells in 24 well plates were Infected with 200 uL of CMV dilutions at 37° C. for 2 h. Following removal of virus inoculums, the infected cells were washed with PBS and overlaid with 0.5% methylcellulose in culture medium. We then treated CMV Infected cells for 3 days at 37° C. with different concentrations of PA and FA. The culture medium was collected for titration of released virus and the cells were harvested for titration of intracellular virus. The results showed that both PA and FA can inhibit CMV replication with EC₅₀ of approximately 230 ug/mL for PA and approximately 60 ug/mL for FA.

### EXAMPLE 7

### Systemic Administration

A systemic preparation of picolinic acid, its derivatives or analogs, containing approximately 1% to 100% active ingredient, may be administered orally, intravenously, intramuscularly or by any acceptable route for the treatment of viral systemic infections such as Dengue. For example, picolinic acid prepared in 00 gelatin capsules at 500 mg per capsule has been shown to be effective in the control in humans of certain lymphomas, and viral diseases such as Herpes simplex labialis, herpes zoster and papilloma viruses. Likewise, an injectable form may be prepared.

As set out above, the safe and effective daily systemic dose may range from 250 mg to 6 grams a day, and the most preferred dose being 500 mg to 2000 mg per day.

TABLE 12

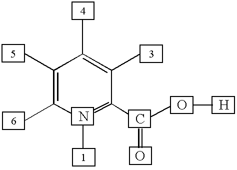

Group a: High Probability of Availability, quantity and Purity

| 1 | 2 | 3 | 4 |
|---|---|---|---|
| CO2H | H | H | H |
| H | CO2H | H | H |
| H | H | CO2H | H |
| H | H | H | CO2H |
| CO2H | H | CO2H | CO2H |
| CH3 | H | H | H |
| H | H | H | CH3 |
| H | Et | H | H |
| H | H | n-Propyl | H |
| H | H | n-Bu | H |
| H | H | Et | H |
| H | H | n-Heptyl | H |
| H | H | 4-n-Hexylcyclohexyl | H |
| H | H | cyclohexyl | H |
| H | t-BuOCONHCH2 | H | H |
| H | H | H | F |
| H | Cl | H | H |
| H | H | H | Cl |
| Cl | H | Cl | H |
| Cl | H | H | Cl |
| Cl | Cl | Cl | Cl |
| H | H | H | Br |
| I | H | H | H |
| OH | H | H | H |
| H | H | OH | H |
| H | H | H | OH(═O) |
| nPrO | H | H | H |
| NH2 | H | H | H |
| H | NH2 | H | H |
| H | H | NH2 | H |
| H | NO2 | H | H |
| C6H5CH25 | H | H | H |
| CONH2 | H | H | H |
| H | H | CO2CH3 | H |
| H | H | H | CO2CH3 |
| H | H | H | CO2Et |
| C8H5CO | H | H | H |
| H | OH((C═O) | H | CO2H |
| Cl | NH2 | Cl | Cl |
| H | H | NO2 | CO2H |
| CO2H | H | H | CH3 |
| H | C6H5 | CO2H | H |
| H | CO2H | CO2H | CH3 |

Antiviral Therapy with Biological Response Modifiers and Zinc Finger Proteins

Because of the intimate association between RNA or DNA viruses with the DNA and RNA molecules of their host cell's synthesizing machines, the development of antiviral agents followed the chemical path of synthesizing compounds against apparently specific targets provided by these molecules. The results of “nucleic acid approach”, although successful in some instances showed that they due to the complexity of the host response of viruses that include much more that nucleic acid was rather simplistic and empirical for the most part. Thus, these agents were either marginally effective or toxic with little benefits for the patients.

The discovery by Isaacs and Lindenmann (1957) of interferon (IFN), a low molecular weight protein produced by cells in response to cells Infected with pathogenic viruses, changed the dogma of interfering with nucleic acids that essentially are common for both viruses and host cells in its chemical properties. There are three classes of interferons (alpha, beta and gamma) with different functions and properties and they inhibit a wide range of viruses and are effective on some of them event to curing a few viral diseases. However, they are clearly not a universal panacea because the resistant of host cells to viruses comprises a myriad of molecular mechanism that act in concert.

A growing body of evidence suggests that the most effective targets to disintegrate viruses reside in proteins that are essential for replication of viruses in host cells. These proteins contain essential domains for viral survival denoted Zinc Finger Domains, that when disrupted by disrupting agents such as picolinic acids and the derivatives thereof, have their ZPF deactivated. The Zn²⁺ is necessary to provide the ZFPs a stable configuration to function as transcription factors. Even a single mutation in the zinc finger biding domain, and in particular the cysteines or histidines residue of the ZF domain, to which Zn²⁺ coordinately binds, results in a protein incapable of functioning. Under these conditions the Zn²⁺ cannot bind the C and H amino acids and maintain the active ZFP configuration. Up to the present time all mutations in the ZFD have been found to be lethal for the virus. During this process of ZFP mutation, the protein is denature, linearized, exposed to host cells proteolytic enzymes and the host cell enters in apoptosis, eliminating both the virus and the virally Infected cell. (For details, please see Fernandez-Pol et al, Anti-cancer Research 2001).

Because picolinic acid acts in potent concert with IFNs, cytokines, chemokines and hundreds of growth factors which act to protect Infected host cells, it is highly likely that the use of biological response modifier, such as picolinic acid and derivatives thereof, will be able to prevent, control or attenuate replication of Dengue viruses in human hosts (and numerous other viruses) in combination with IFNs, chemokines, and cytokines. These results have been amply documented in previous sections of the instant invention.

Culture Monocytes or Macrophages that Support Replication of Dengue Viruses

Dengue, widespread through tropical and subtropical regions of the world, puts an estimated 2.5 billion people at risk for dengue vascular permeability, Dengue hemorrhage fever, Dengue shock syndrome (DSS), and involvement of the liver accompanies DHF and/or DSS. Culture monocytes or macrophages support replication of dengue viruses. These Australian investigations have established a well defined cell culture of macrophages and Infection system for DEN-2 that produces high titers of viral growth, and permanent Infection with DEN-2 in relevant human primary cells in the absence of enhancing antibody. In this model, the production of intracellular virus paralleled that for secreted virus (Pryor et al 2001).

Macrophages are ubiquitous cells critical for host defense (Varesio et al., 1985). Metchnikoff's original report in the last portion of the 19^{th} century first indicated that phagocytes are the body's primary detectors of foreign invaders. Initially, the functions of macrophages have been studied at the cellular level. Presently, macrophage study includes powerful genetic engineering techniques (Vareisio et al.) and isolation of recombinant growth factors which has found a list of macrophage secretor products and the interaction with metabolites of L-tryptophan, such as picolinic acid, that is impressive. This modern study has lead to a new approach of antiviral therapy; often the prevention, therapy and attenuation of Dengue viruses.

### EXAMPLE 8

### Anti-Plasma Leaking of Nicotinamide and Antithrombotic Effects of Nicotinic Acid: Potentially Useful Pharmacological Actions of these Agents to Treat Dengue Hemorrhagic Fever (DHF) and Dengue Shock Syndrome (DSS)

Nicotinic acid, also known as niacin, functions in the body after conversion to either nicotinamide adenine dinucleotide (NAD) or nicotinamide adenine dinucleotide phosphate (NADP). Nicotinic acid functions in both NAD and NADP in the form of amide, nicotinamide. The chemical structures of NAD and NADP are as follows in Table 13:

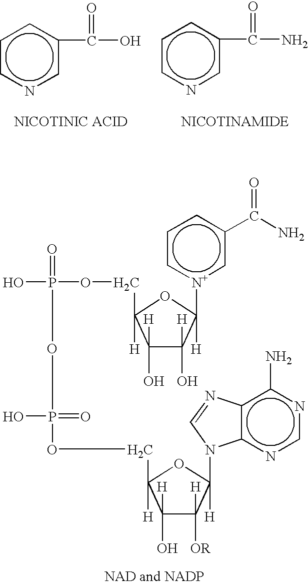

Nicotinic acid and nicotinamide are identical in their physiological functions. However, nicotinic acid is unable to be converted directly to nicotinamide, which is originated from the metabolism of NAD.

Niacin has multifarious lipoprotein and anti-atherothrombosis effects that improve endothelial cell function, reduces inflammation, and decreases thrombosis. It is the most effective agent for increasing high-density lipoprotein cholesterol. Niacin reduces blood viscosity through a variety of mechanisms, thus improving blood flow and perfusion through stenotic or thrombotic segments of the veins and arteries. Niacin preserves glycolysis during periods of ischemia, and thus may accelerate the recovery functions of affected organs (Rosenson, RS, 2003).

Fernandez-Pol et al., in 1977 (Proc. Natl. Acad. of Sci. USA) showed that nicotinamide in tissue culture added to SV40-virally transformed cells have a pronounced effect on cell shape: the SV40-C become very flat and increase the adhesiveness between them in petri dishes, to the point of forming a mosaic of tightly adherent cells. This indicates that Nicotinamide has the capacity to increase the adhesive cell surface proteins, indicating that the monolayer, even at high cell densities, prevents the free passage of culture media to the bottom of the cells which are strongly attached to the plastic petri dish. These observations suggested to the Inventors that nicotinamide could be useful to prevent leakage of culture media, or if in an intact animal, plasma which will prevent edema and extravasation of fluids. The Inventors believe that those are the bases for the use of nicotinamide in DSS in which extravasation of blood fluids causes the disequilibrium in the blood-extracellular compartment due to increased permeability. Thus, nicotinamide may be a useful pharmacological agent to treat dengue DSS.

It has been recently shown that niacin (Tavintharan, S. et al., 1977) decreases atherothrobotic events in vein and arteries by lowering prothrombotic factors such as PAI-1. It also reduces cell adhesion molecules (CAM) (as does nicotinamide). The results showed that treatment with niacin suppressed PAI-1 and ICAM-1 levels in the cells tested (HepG2). Thus, niacin at pharmacological concentrations can be used for the prevention and/or treatment against thrombotic disease in patients with DSS. In addition to the antithrombotic effects, niacin inhibits vascular oxidative stress, and monocyte adhesion to human aortic endothelial cells (Ganji, SH et al., 2008). In pharmacological doses, niacin reduces stroke and artherosclerosis. These effects are mediated by the actions of niacin on lipoproteins. Niacin increases nicotinamide adenine dinucleotide phosphate (NAD(P)H) levels by 54% and reactive oxygen species (ROS) by >50%, vascular adhesion molecules (VCAM-I) by 80%, and very significantly monocyte chemotactic protein-I (MCP-I) secretion by 34 to 124%. TNF-alpha is also reduced and adhesion decreases. These studies indicate that niacin inhibits: endothelial vascular inflammation, ROS production and inflammatory cytokine production. These are key events in the production of damage to the endothelium of arteries, atherogenesis, and subsequent thrombosis in the damage areas by LDL. These results demonstrate that in addition to the anti-atherogenic properties, niacin has potent anti-inflammatory vascular properties that could prevent the development of lethal thrombosis.

Thrombocytopenia is frequently associated with dengue virus infection in humans. Basu, A et al. have demonstrated that Dengue 2 virus inhibits in vitro megakaryocytic (MK) colony formation and induces apoptosis in thrombopoieting-inducible megakaryocytic differentiation in blood CD34+ cells. In summary, dengue 2 viruses can inhibit in vitro megakaryopoiesis (production of MK cells), and infect the MK, to such an extent that induce apoptosis of MK cells, depriving the human body from platelets that would prevent uncontrollable bleeding. The inventors believe that these pathological events of the virus play a key role in the etiology of the thrombocytopenia in Dengue thrombosis and plasma fluid leakage syndromes (DHF and DSS). The inventors consider that prevention of megakaryocytic destruction by use of picolinic acid in combination with prevention of DHF and DSS with niacin or nicotinamide can be effective in controlling these deathly syndromes.

1-Methylnicotinamide (MNA), a metabolite of nicotinamide, also exerts anti-thrombotic activity which is mediated by cyclooxygenase-2/prostacyclin pathway (Chlopicki, S, et al. Br. J. Pharmacol, 2007). N-methylnicotinamide inhibits arterial thrombosis in experimental animals. MNA is effective in animals with thrombolysis, intra-aterial thrombus formation and in venous thrombosis. When compared with compounds used in standard clinical practice, MNA was more effective in inhibiting platelet-dependent thrombosis. It is conceivable that nicotinic acid or MNA can be effective in preventing the plasma and blood leakage to tissues and the subsequent thrombosis that may result by the stagnant blood and sera that accumulates extravascularly.

In animal experiments, Nicotinamide (NA) can function as a cytoprotectant for acute and chronic neurodegenerative disorders and viral encephalopathies. NA prevents neuronal and vascular cell injury, apoptosis, and maintains phosphatidyl serine membrane asymmetry, which prevents inflammation, cellular phagocytosis and vascular thrombosis, Maiese, K & Chong, Z Z, in 2003 (Trends Pharmacol. Sci.). Furthermore, NOA maintains high mitochondrial membrane potentials, increasing cellular energy which remains independent of cytoplasmic intracellular pH and growth factor activated protein kinases. Cysteine protease activity, during cerebral vascular endothelial cell injury induced by viruses undergoes modulation by NA. Considering the association of dengue fever with clinical manifestations of encephalopathy, neurologic disorders, peripheral neuropathy, polyneuritis, and Bell's palsy, the Inventors propose that the neurological manifestations of dengue hemorrhagic fever can be treated, or attenuated, with appropriate doses of NA ranging from about 500 mg to about 6 grams for a 70 kg weight patient.

In conclusion, the demonstration of the co-stimulatory effects of picolinic acid, IFN-gamma and other critical cytokines and chemokines has made it possible for picolinic acid to act as an autocrine mediator in the induction of antiviral activity against numerous viruses including Dengue. The production of picolinic acid by macrophages in conjunction with IFN-gamma clearly shows that PA and IFN-gamma can induce apoptosis in virally infected cells. Furthermore, the actions of PA on ZFP render them unable to replicate DNA or RNA viruses (Fernandez-Pol, 2001).

From the aforementioned description, a method to control dengue viruses in humans by picolinic acid and derivatives thereof has been described. The method is uniquely capable of stimulating development and introduction of macrophages in the vicinity of the site of virus introduction. The method reduces the release of intercellular fluids and byproducts into the intra-cellular environment within the body of an animal or a person. The method and its various steps and components may be manufactured from many materials, including but not limited to, polymers, amino acids, minerals, vitamins, compounds thereof, and substitutions.

As such, those skilled in the art will appreciate that the conception, upon which this disclosure is based, may readily be utilized as a basis for the designing of other structures, methods and systems for carrying out the several purposes of the present invention. Therefore, the claims include such equivalent constructions insofar as they do not depart from the spirit and the scope of the present invention.

We claim:

1. A method of treating or preventing an arbovirus in an animal or a human, the virus being mediated by a cellular or viral metalloprotein within the cell infected by the virus or present in the viral core, said method comprising:
administering at least one pharmacological agent systemically in a therapeutically effective amount, said agent including picolinic acid and its derivatives, and a biological response modifier including interferons, chemokines or cytokines;
delivering said at least one agent, to the infected cells of an animal or a human, to inactivate the metalloprotein; and,
said at least one agent belonging to the family of picolinic acid and having the following structure: 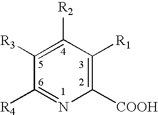
or a pharmacologically acceptable salt thereof;
wherein R1 is selected from the group consisting of butyl group, carboxyl group, ethyl group, hydrogen, isobutyl group, isopentyl group, isopropyl group, methyl group, neopentyl group, pentyl group, propyl group, secondary butyl group, and the tertiary butyl group;
wherein R2 is selected from the group consisting of butyl group, carboxyl group, ethyl group, hydrogen, isobutyl group, isopentyl group, isopropyl group, methyl group, neopentyl group, pentyl group, propyl group, secondary butyl group, and the tertiary butyl group;
wherein R3 is selected from the group consisting of butyl group, carboxyl group, ethyl group, hydrogen, isobutyl group, isopentyl group, isopropyl group, methyl group, neopentyl group, pentyl group, propyl group, secondary butyl group, and the tertiary butyl group; and,
wherein R4 is selected from the group consisting of butyl group, carboxyl group, ethyl group, hydrogen, isobutyl group, isopentyl group, isopropyl group, methyl group, neopentyl group, pentyl group, propyl group, secondary butyl group, and the tertiary butyl group.

2. The virus preventing and treating method of claim 1 further comprising:
increasing antiviral activity of macrophages through said at least one agent resulting in a synergistic action between picolinic acid, interferons, cytokines and chemokines, preventing viral replication; and,
contacting the zinc in the nucleocapside zinc finger metalloprotein of the virus and dissociating and ejecting the zinc from the zinc finger domains, rendering the virus unable to replicate;
wherein the viral proteins disintegrate by the action of macrophage proteolytic enzymes, simultaneously destroy the virus and cause apoptosis of macrophages.

3. The virus preventing and treating method of claim 1 wherein said method is effective against Arbovirus including Togaviridae and Flaviviridae.

4. The virus preventing and treating method of claim 3 wherein said method is effective against Flaviviridae including Sindbis virus.

5. The virus preventing and treating method of claim 3 wherein said method is effective against human Dengue virus 1, 2, 3, or 4 serotypes.

6. The virus preventing and treating method of claim 3 wherein said method is effective against human hepatitis C virus.

7. The virus preventing and treating method of claim 3 wherein said method is effective against human hepatitis B virus.

8. The virus preventing and treating method of claim 3 wherein said method is effective against Pestivirus bovine viral diarrhea virus.

9. The virus preventing and treating method of claim 3 wherein said method is effective against human cytomegalovirus.

10. The virus preventing and treating method of claim 3 wherein said method is effective against varicella-zoster virus.

11. The virus preventing and treating method of claim 3 wherein said method operates systemically.

12. The virus preventing and treating method of claim 3 further comprising:
said at least one agent increasing antiviral activity of macrophages resulting in a synergistic action between picolinic acid and one of nicotinamide or nicotinic acid.

13. The virus preventing and treating method of claim 3 further comprising:
said at least one agent increasing antiviral activity of macrophages resulting in a synergistic action between picolinic acid, nicotinamide, and nicotinic acid.

14. The virus preventing and treating method of claim 3 further comprising:
said at least one agent reducing damage to the nervous system of a human or an animal.

15. The virus preventing and treating method of claim 14 wherein said at least one agent functions as a cytoprotectant and reduces the damage to the cells of a human or an animal from excessive production of fluids, chemokines, and cytokines.
